(19) United States
(12) Plant Patent Application Publication
(10) Pub. No.: US 2017/0094854 P1
(43) Pub. Date: Mar. 30, 2017

(54) Apple tree rootstock 'G.213'

(50) ;

(71) Applicants: Cornell University, Ithaca, NY (US); The United States of America, as Represented by the Secretary of Agriculture, Washington, DC (US)

(72) Inventors: Gennaro Fazio, Geneva, NY (US); Herb Aldwinckle, Geneva, NY (US); James Cummins, Alcoa, TN (US); Terence Robinson, Geneva, NY (US)

(73) Assignees: Cornell University, Ithaca, NY (US); The United States of America, as Represented by the Secretary of Agriculture, Washington, DC (US)

(21) Appl. No.: 14/756,708

(22) Filed: Sep. 30, 2015

(51) Int. Cl. A01H 5/00

(52) U.S. Cl. PLT/174

(57) ABSTRACT

The present invention relates to a new and distinct variety of apple tree Malus domestica×Malus robusta hybrid named ‘G.213.’ ‘G.213’ is useful in that it can be propagated clonally and used as a rootstock or root system for apple trees as well as for interstems of apple trees. The new variety is a dwarfing rootstock that is resistant to fire blight (Erwinia amylovora) and crown rot (Phytophthora cactorum).

### STATEMENT REGARDING FEDERAL FUNDING

This invention was made with United States government support under a United States Department of Agriculture-Agricultural Research Station Cooperative Research and Development Agreement, Sponsor's Contract Number 58-3K95-4-1668-M. The government has certain rights in the invention.

Genus and species: Malus domestica×Malus robusta hybrid.

Variety denomination: ‘G.213’.

### BACKGROUND OF THE NEW PLANT AND COMPARISONS

I. Field & Utility Summary

The present invention relates to a new and distinct variety of apple tree. The apple tree is particularly useful in that it can be propagated clonally and used as a rootstock or root system for apple trees as well as for interstems of apple trees. The new variety is a dwarfing rootstock that is resistant to fire blight and crown rot. It is precocious and highly productive.

II. Cultivation Summary

‘G.213’ originated from a planned cross in 1976 in Geneva, N.Y.

III. Comparisons

The seed parent Malus domestica hybrid ‘Ottawa 3’ is a dwarfing rootstock, i.e., trees grown on this rootstock are 25 to 35 percent the size of a standard self-rooted seedling apple tree. ‘Ottawa 3’ is well known to induce precocity to the scion (i.e., the ability to induce early reproductive development on grafted scions) and has good yield efficiency (i.e., the ability to produce many apples per square centimeter of trunk). ‘Ottawa 3’ produces no spines in stool bed clonal propagation, it is winter hardy, but susceptible to the woolly apple aphid (Eriosoma lanigerum), to fire blight (Erwinia amylovora), and to the specific replant disease complex.

The pollen parent Malus robusta ‘Robusta 5’ is a non-dwarfing rootstock, i.e. trees on this rootstock grow to the same size of a standard self-rooted apple tree. ‘Robusta 5’ does not induce precocity to the scion and is not yield efficient. Juvenile plants and young stool propagules of ‘Robusta’ 5 produce many spines. ‘Robusta 5’ is resistant to fire blight and powdery mildew (Podosphaera leoucotricha), and is immune to the woolly apple aphid. ‘Robusta 5’ breaks buds very early in the spring and is winter hardy. Further, ‘Robusta 5’ has shown tolerance to the specific replant disease complex.

The ‘G.213’ apple rootstock of the present invention has a combination of qualities that distinguishes it from its parental plants (i.e., ‘Ottawa 3’ and ‘Robusta 5’). For example, although ‘Ottawa 3’ is susceptible to the woolly apple aphid, ‘G.213’ has resistance to it. In addition, ‘G.213’ has a yield efficiency similar to ‘Ottawa 3’. ‘G.213’ has dwarfing properties derived from ‘Ottawa 3’, and belongs to the same dwarfing vigor class as its dwarfing parent (i.e., ‘Ottawa 3’), allowing trees grafted on this rootstock to grow 25 to 35 percent of standard self-rooted apple tree seedlings. Specifically, ‘0.213’ is in the same dwarfing vigor class as industry standard ‘Malling 9’. ‘G.213’ shares further similarities with ‘Ottawa 3’ in that they both induce precocity to the scion and are winter hardy.

With regard to its ‘Robusta 5’ parent, ‘0.213’ is distinguishable in that it is a dwarfing rootstock and is yield efficient, while ‘Robusta 5’ is not. However, like ‘Robusta 5’, ‘G.213’ is resistant to fire blight and the woolly apple aphid (Eriosoma lanigerum), and produces some spines (sylleptic small branches) at the base of clonally propagated stool bed shoots. In addition, ‘G.213’ has been shown to have some tolerance to the replant disease complex.

As discussed above, ‘G.213’ is most similar to ‘Malling 9’ in terms of dwarfing class. However, ‘G.213’ is resistant to fire blight and wooly apple aphid whereas ‘Malling 9’ and other rootstocks in its market class are not.

IV. Breeding History

In the spring of 1976, pollen from a Malus robusta ‘Robusta 5’ apple tree was applied to emasculated flowers of a Malus domestica hybrid ‘Ottawa 3’ in Geneva, N.Y. In the fall of 1976, seeds resulting from this pollination were extracted from mature fruit derived from this cross. In the winter of 1976-77, the seeds were stratified and planted in large flats under conditions effective to germinate seeds and obtain seedlings. When germinated seedlings were about 2.5 cm tall they were inoculated with a mixture of isolates of the fungus Phytophthora cactorum (the causal agent of crown and root rots). The flats were flooded to mid-hypocotyl level and kept at 23° C. for one week. Surviving seedlings were transplanted into individual pots.

In the summer of 1977, each of the transplanted seedlings was inoculated with approximately 106 colony forming units of the Ea 273 strain of the fire blight bacterium Erwinia amylovora by inserting a 26-gauge hypodermic syringe needle into the shoot tip. The seedling designated as #213 was one of the survivors of this battery of inoculations from the same cross. All the surviving plants were transplanted to the nursery field (Geneva, N.Y.) in the fall of 1977 and allowed to grow side shoots for propagation/evaluation.

In 1980, ‘G.213’ was evaluated for rooting ability, lack of spine production, and low root brittleness in a layering bed (stool bed). In 1983, 4 finished trees with ‘G.213’ rootstock were planted in a first test orchard (Geneva, N.Y.) with Malus domestica cv. ‘Northern Spy’ grafted onto this rootstock as the scion cultivar. This rootstock performed well (top 20% of many rootstocks tested) in these first test trials, and during 1990-1993 more material was propagated by stool bed and nursery to be entered into new trials at a second test orchard (Geneva, N.Y.) with Malus domestica cv. Empire as the scion cultivar. The ‘G.213’ rootstock performed well with all the scion cultivars that were tested. In August of 2002, several rootstock liners of ‘G.213’ were budded with 15 different scion cultivars to test graft union compatibility the test results showed that ‘G.213’ was compatible with all cultivars tested.

V. Asexual Reproduction

Asexual reproduction of the ‘G.213’ apple rootstock has been achieved using the traditional method of clonally propagating apple rootstocks. In particular, the original seedling of the ‘G.213’ apple rootstock was planted in the field (Geneva, N.Y.) and allowed to develop into a “mother plant.” The ‘G.213’ mother plant was then used to obtain rooted liners using conventional layering procedures. The resulting liners were then planted in a row to generate a layering stool bed (also referred to as the “mother stool bed”). The living tissues (i.e. leaves, stems, roots, buds, and spines) of the mother stool bed were observed to be identical to secondary and tertiary stool bed plants. In addition to conventional layering, the ‘G.213’ apple rootstock variety has been asexually reproduced by root cuttings, by budding and grafting onto seedling and clonal rootstocks, and by in vitro culture. Asexual reproduction by layering, root cuttings, budding, grafting, and tissue culture in Geneva, N.Y. has shown that the claimed plant reproduces true to type through successive generations of asexual reproduction.

VI. Stability

Observations of trees from these propagations indicate that all trees have proven true to type and identical in all appearances to the original tree.

### SUMMARY

The present invention relates to a new and distinct variety of apple tree rootstock named ‘G.213.’ The ‘G.213’ apple tree rootstock is a dwarfing rootstock that is resistant to fire blight (Erwinia amylovora) and crown rot (Phytophthora cactorum). The ‘G.213’ rootstock is useful in that it can be propagated clonally and used as a rootstock or root system for apple trees as well as for interstems of apple trees. While ‘G.213’ rootstock is a hybrid from a cross between ‘Ottawa 3’ and ‘Robusta 5’, it is distinct from its parent cultivars in terms of dwarfing and disease resistance. The apple tree rootstock ‘G.213’ produces grafted trees that are 25 to 35 percent the size of self-rooted standard apple trees, it induces good precocity on the scion, and is tolerant to the replant disease complex.

### BRIEF DESCRIPTION OF THE PHOTOGRAPHS

The new apple tree ‘G.213’ is illustrated by the accompanying photographs. The colors shown are as true as can be reasonably obtained by conventional photographic procedures.

FIG. 1 shows non-dormant shoot (including, spines, buds and bark).

FIG. 2 shows a close-up view of a live shoot (including expanding leaves).

FIG. 3 shows the adaxial (upper) lamina surface of mature leaves.

FIG. 4 shows the abaxial (lower) lamina surface of mature leaves.

FIG. 5 shows the mature fruit.

FIG. 6 shows shoots on a clonal propagation stool bed.

FIG. 7 shows 8 year old trees.

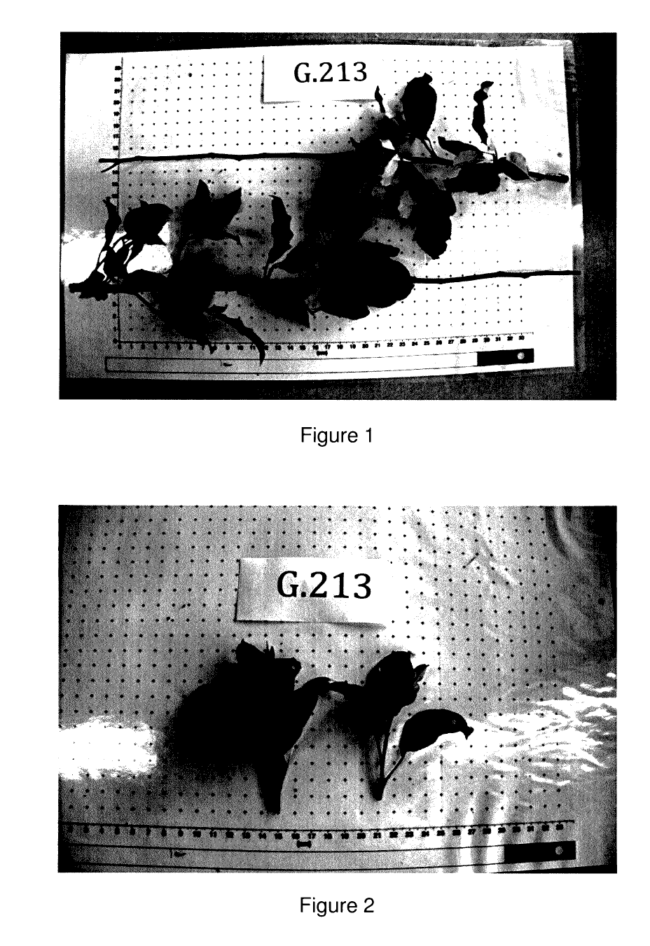

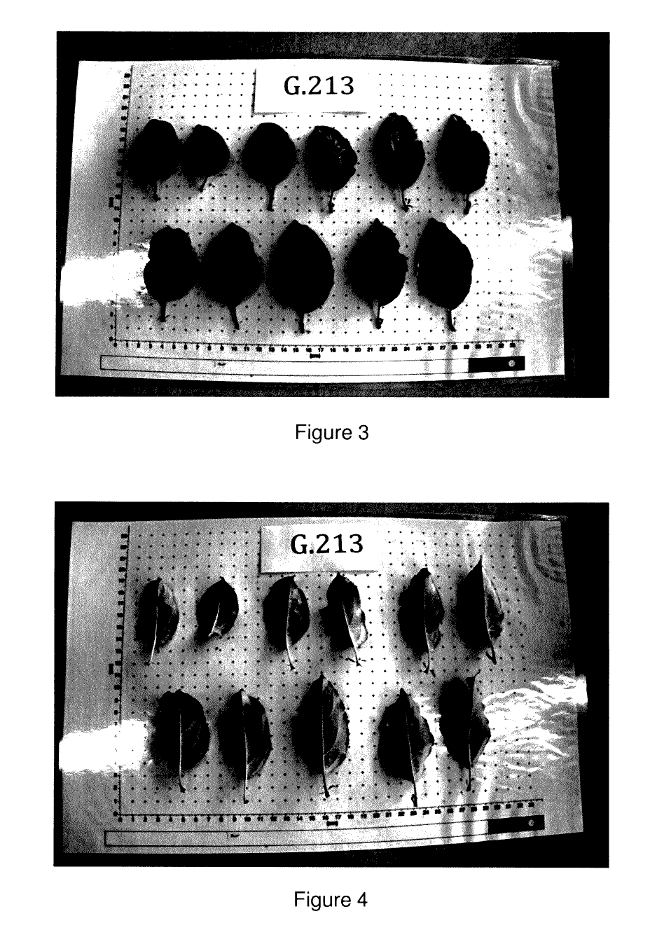

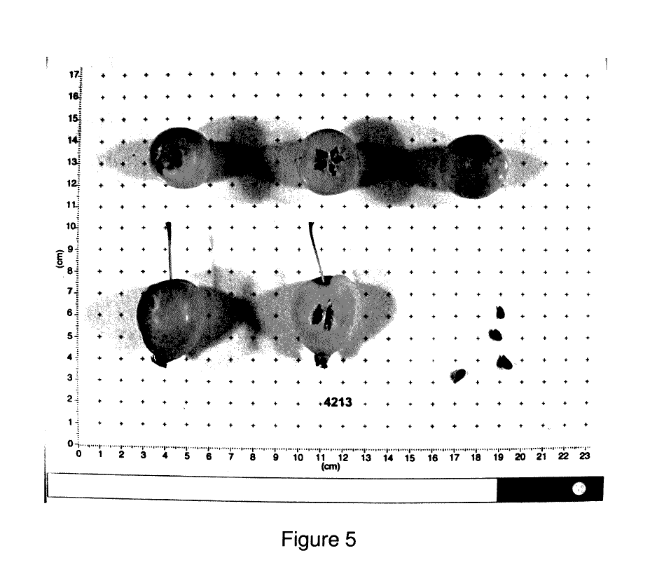

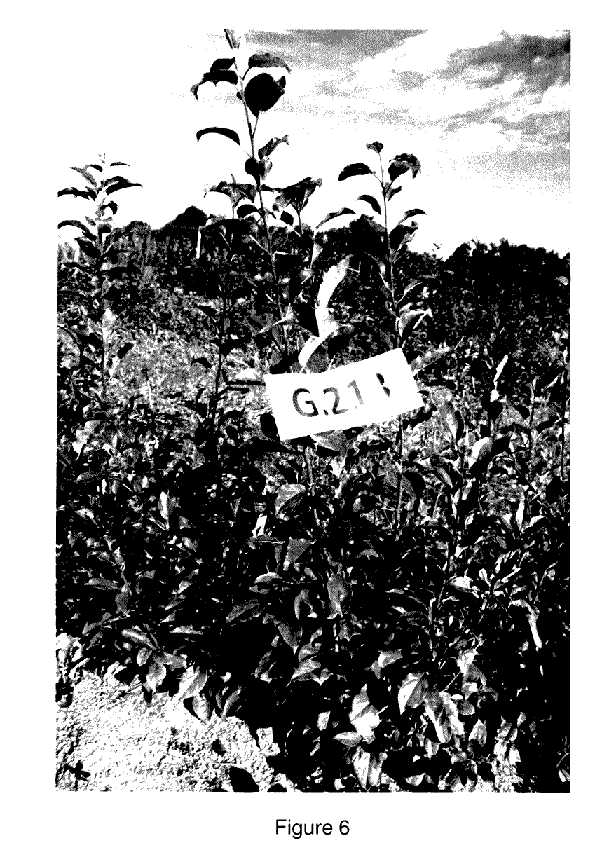

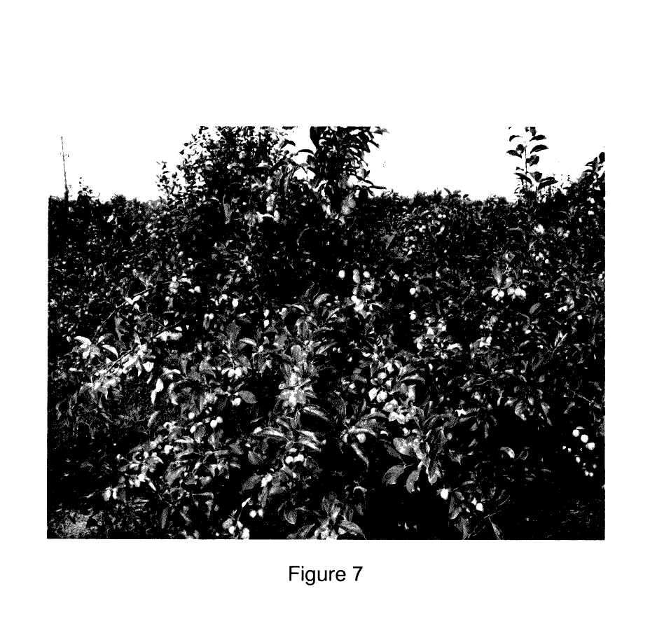

### DETAILED BOTANICAL DESCRIPTION OF THE PLANT

The following detailed descriptions set forth the distinctive characteristics of ‘G.213’. The data which define these characteristics were collected from asexual reproductions carried out in Geneva, N.Y. Color references are to The Royal Horticultural Society Colour Chart (R.H.S.), 2001 edition. Botanical descriptions follow the Manual of Cultivated Plants (Bailey, 1949).
Tree:
   Habit.—A self-rooted tree of ‘G.213’ is a small shrub typically standing about 1.5-2 meters tall by about 2 meters wide when 7 years old. There is no single dominant trunk. Instead there are few shoots arising from the crown. Very few suckers (i.e., new shoots emerging from below ground) are produced. Liners planted in the nursery stop apical growth mid season.
   Productivity.—In an intermediate trial performed in Geneva, N.Y., the ‘G.213’ rootstock received the cultivar ‘Empire’ (Malus domestica) as the scion and was compared to the Malus domestica check rootstocks M.9 EMLA, M.26 and M.7. ‘G.213’ was shown to have statistically higher (p≦0.05) yield efficiency (kg yield/cm² trunk cross sectional area) than all the check rootstocks.
   Precocity.—Scion cultivars budded on ‘G.213’ exhibit the same precocity as those budded on M.9.
   Fertility (Fecundity).—The ‘G.213’ plant produces flowers and fruits regularly.
Dormant shoots (buds and bark):
   Dormant mature shoots.—Color: Greyed-Orange (RHS 175C) where exposed to full sunlight grading to Greyed-Orange (RHS 173B) with diminished light exposure. Texture: Very light pubescence which gradually disappears in older tissues. Size: 30-60 cm long; may have some spines.
   Axillary buds.—Size: 2-3 mm long and 3 mm wide with little pubescence. Shape: Obtuse, sessile, somewhat appressed and flattened. Texture: Some pubescence.
   Bark on three-year-old shoots.—Color: Greyed-Brown (RHS 199A). Lenticels: Color: Greyed-Orange (RHS 163B). Size: 0.3-0.4 mm in diameter. Quantity: 1-2 lenticels per cm².
Leaves:
   Mature leaves.—Leaf arrangement: Alternate. Shape: Simple, recurved, oblong-ovate. Size: Length: 95 mm. Width: 35 mm, at the widest point. Laminae: Somewhat wavy. Apex: Acuminate. Base: Nearly symmetrical rounded. Margin: Acutely serrated, with about 6 serrations per cm. Upper surface: Color: Green (RHS 137C). Texture: Glabrous and translucent. Lower surface: Color: Green (RHS 147C). Texture: Somewhat pubescent. Venation: Netted. Leaf poise: 15°-25° from the shoot, depending on shoot orientation. Stipules: Length: 8 mm. Width: 2 mm. Petioles: Diameter: 2 mm. Color: Gradation of Green (RHS 140A) to Greyed-Red (RHS 179A) depending on low or high exposure to light.
Flowers:
   Habit.—Flowers borne on spurs, shoot terminals, and from lateral buds on growth from previous season.
   Flower diameter.—40 mm.
   Fragrance.—None.
   Buds.—Location: Located on spurs and terminals; are mixed, typically producing a truss of 5 to 6 flowers and one bourse shoot. Shape: Lateral buds are obtuse, sessile, and somewhat appressed. Size: Length: 4 mm. Width: 3 mm. Habit: Buds near the base of the shoot of the previous season usually produce 3 to 5 flowers and a single short shoot; mid-shoot buds may have 2-4 flowers; and more distal buds are usually vegetative.
   Petals.—Size: Length: 25 mm. Width: 18 mm. Shape: Spatulate Apex: Obtuse Margin: Smooth Texture: Smooth Color: Closed petals: Red (RHS 54A). Open petals: White (RHS 155D) with Red (RHS 54A), striations in some of the petals.
   Reproductive organs.—Pistils: Length: 6 mm. Color: Yellow-white (RHS 145B). Stamens: Length: 4 mm. Color: White (RHS 155D). Anther color: Yellow-Brown (RHS 167D).
Fruit:
   Mature fruit.—Size: Height: 35-40 mm. Diameter: 30-35 mm. Shape: Obloid. Color: Partial Orange skin (RHS 2aA) with Red (RHS 46C) blush overtones depending on the exposure to the sun. Sepals: Persisting on a very protruding calyx. Flesh: Taste: Astringent (not meant for consumption). Color: Yellow-Orange (RHS 20C). Seed: Color: Greyed-Red (RHS 179A), translucent. Shape: Tear drop shape. Size: Length: 50-60 mm. Diameter: 25-35 mm at the widest point. Number: Generally five seeds per fruit.
Disease resistance: As described above, the ‘G.213’ rootstock of the present invention exhibits resistance to fire blight. The percent lesion measured after inoculation of potted liners in the greenhouse using four different strains of E. amylovora was negligible for two of the strains and moderate for the other two, indicating a specific resistance to the bacterium. The ‘G.213’ rootstock, having survived the inoculation with crown and root rot, is also considered resistant to crown and root rots caused by Phytophthora cactorum. G.213 seems to be susceptible to powdery mildew.

We claim:

1. A new and distinct variety of apple tree named ‘G.213’ as shown and described herein.
